(19) United States
(12) Patent Application Publication
(10) Pub. No.: US 2006/0111758 A1
(43) Pub. Date: May 25, 2006

(54) Apparatus and methods for replacement of files in a receiver of an in-vivo sensing system

(76) Inventors: Micha Nisani, Nesher (IL); Pesach Pascal, Nesher (IL)

Correspondence Address: PEARL COHEN ZEDEK, LLP, 1500 BROADWAY 12TH FLOOR, NEW YORK, NY, 10036, US

(21) Appl. No.: 10/992,815

(22) Filed: Nov. 22, 2004

(51) Int. Cl. A61N 1/08

(52) U.S. Cl. 607/59

(57) ABSTRACT

Methods and systems according for replacement of an operational file in a receiver of an in vivo sensing device with a file received from an apparatus external and/or remote from the receiver. An operational file may be replaced by an updated version.

### FIELD OF THE INVENTION

The present invention relates to an apparatus and method for software and hardware replacement. More specifically, the invention relates to updating a receiving and/or recording unit of an in vivo sensing system.

### BACKGROUND OF THE INVENTION

In-vivo devices, such as, for example, capsules, may be capable of gathering information regarding a body lumen while inside the body lumen. Such information may be, for example, a stream of images of the body lumen and/or measurements of parameters that are of medical concern, such as, for example, pH. The sensing device may transmit the gathered information via a hard-wired or wireless medium, and the gathered information may be received by a receiver/recorder, that may record the gathered information. The recorded information may be sent from the receiver/recorder to a work station to be analyzed and/or displayed.

The receiver/recorder may be operated by, for example, health care professionals and technicians, in a hospital, or another health facility. It may be desired to minimize “down time” of the receiver/recorder, in which the receiver/recorder is not operative.

### SUMMARY OF THE INVENTION

Methods and systems according to embodiments of the present invention enable replacement of an operational file in a receiver with a file received from an apparatus external and/or remote from the receiver. For example, updated versions of operations files may thus be installed in an in vivo sensing system, according to embodiments of the invention.

The term “receiver” may be meant to also include a recorder, but does not have to include a recorder. According to some embodiments, the operational file may be an operational hardware configuration file or a software file.

A system according to some embodiments of the invention may include a sensing device, such as an imaging device, or a device for sensing physiological parameters of a body lumen such as, pH, temperature, pressure, electrical impedance, etc., a receiver that can receive sensed data from the sensing device and a workstation that may process and/or display sensed data (e.g., image data). According to embodiments of the invention the workstation may receive a file, e.g. an updating file, and it may send the received file to the receiver. According to some embodiments the system may include a processor. The processor may, according to some embodiments, check whether a condition (or set of conditions) for replacing an operational file of the system with the received file is fulfilled.

According to some embodiments the receiver may contain two or more modules or memories in which operational files and/or received files can be saved, optionally temporarily.

According to one embodiment the receiver may be powered by a power source and a condition for replacing an operation file with a received file may be, for example, that the power source stores energy that is sufficient for replacing the operational file with the received file.

A method according to an embodiment of the invention may include the steps of: replacing an operational file of a receiver of an in-vivo sensing system with a received file, the received file being received from an apparatus that is external to the receiver storing said received file in a program memory. The method may include storing the received file in a storage (e.g., a temporary storage) prior to replacing the operational file with said received file. According to other embodiments the method may include detecting/correcting errors in the received file prior to replacing the operational file with said received file.

According to embodiments of the invention an article is provided which may include a computer-readable storage medium having stored thereon instructions that, when executed by a processor, result in deciding whether to replace an operational file of a receiver of an in-vivo sensing system with a received file.

According to some embodiments other components may be used for the method of replacement, for example, the replacement need not be executed in the receiver but rather in the workstation or in any other suitable component of a system.

### BRIEF DESCRIPTION OF THE DRAWINGS

Embodiments of the invention are illustrated by way of example and not limitation in the figures of the accompanying drawings, in which like reference numerals indicate corresponding, analogous or similar elements, and in which:

FIG. 1 is a simplified illustration of an exemplary in-vivo sensing system, including an in-vivo sensing device, a receiver/recorder, a work station and an optional computer, in accordance with some embodiments of the invention;

FIG. 2 is an exemplary simplified block-diagram illustration of a receiver/recorder of an in-vivo sensing system, in accordance with some embodiments of the invention;

FIG. 3 is a simplified flow chart illustration a method to replace an operational software file in a receiver/recorder of an in-vivo sensing system, in accordance with some embodiments of the invention;

FIG. 4 is a simplified flow chart illustration of a method to replace an operational software file in a receiver/recorder of an in-vivo sensing system, in accordance with other embodiments of the invention; and

FIGS. 5 and 6 are a simplified flow chart illustration of a method to replace an operational hardware configuration file in a receiver/recorder of an in-vivo sensing system, in accordance with some embodiments of the invention.

It will be appreciated that for simplicity and clarity of illustration, elements shown in the figures have not necessarily been drawn to scale. For example, the dimensions of some of the elements may be exaggerated relative to other elements for clarity.

### DETAILED DESCRIPTION OF THE INVENTION

In the following detailed description, numerous specific details are set forth in order to provide a thorough understanding of embodiments of the invention. However it will be understood by those of ordinary skill in the art that the embodiments of the invention may be practiced without these specific details. In other instances, well-known methods, procedures, components and circuits have not been described in detail so as not to obscure the embodiments of the invention.

An in-vivo sensing system may include an in-vivo sensing device, such as, for example, a capsule having image capturing capabilities, and a receiver/recorder, to receive a stream of images from the in-vivo sensing device and to store the stream of images in a memory for a later use. In addition, the in-vivo sensing system may include a work station, capable of downloading the stream of images from the receiver/recorder and capable of analyzing and/or displaying the stream of images.

According to some embodiments of the present invention, the receiver/recorder may be capable of downloading a software file from the work station, or from another computer, and to replace an existing software file of the receiver/recorder with the received software file.

According to some other embodiments of the present invention, the receiver/recorder may be capable of downloading a hardware configuration file from the work station, or from another computer, and of replacing an existing hardware configuration file of the receiver/recorder with the received hardware configuration file.

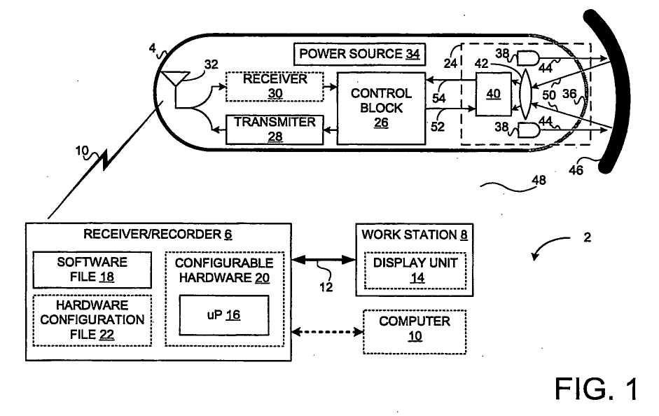

FIG. 1 is a simplified illustration of an exemplary in-vivo sensing system 2, including an in-vivo sensing device 4, a receiver/recorder 6, a work station 8 and an optional computer 10, in accordance with some embodiments of the invention. According to some embodiment of the invention, sensing device 4 may be a capsule, although other configurations are possible and are under the scope of the invention.

As illustrated in the following description, sensing device 4 may be able to gather information, such as, for example, a stream of images, while inside a human patient's body, and may be able to transmit at least that information to receiver/recorder 6 via a wireless or hard-wired medium 10 while inside the patient's body. Receiver/recorder 6 may be able to record information received from sensing device 4.

Work station 8 may be able to receive the recorded information from receiver/recorder 6 via, for example, a wireless or hard-wired medium 12, and may be capable of processing and/or presenting information received from receiver/recorder 6 to an operator. For example, work station 8 may include an optional display unit 14, and may be able to display the stream of images recorded in receiver/recorder 6 on optional display unit 14.

Receiver/recorder 6 may include a processor (uP) 16 to, for example, control, at least in part, the operation of receiver/recorder 6, and may include an operational software file 18, to be executed by uP 16.

According to some embodiments of the invention, work station 8 and/or optional computer 10 may be capable of initiating a process of updating and/or replacing operational software file 18, and receiver/recorder 6 may be capable of performing the process of updating and/or replacing operational software file 18 in a controllable manner and of for example, minimizing risk of damage to receiver/recorder 6 as a result of the process.

Receiver/recorder 6 may optionally include a configurable hardware 20, such as, for example, any combination of one or more field programmable gate arrays (FPGA), and one or more complex programmable logic devices (CPLD). Configurable hardware 20 may be configured to include hardware parts of receiver/recorder 6. For example, and although the invention is not limited in this respect, uP 16 may be a configurable part of configurable hardware 20.

Receiver/recorder 6 may optionally include an operational hardware configuration file 22, wherein the content of operational hardware configuration file 22 may determine the configuration of configurable hardware 20.

According to some embodiments of the invention, work station 8 and/or optional computer 10 may be capable of initiating a process of updating and/or replacing operational hardware configuration file 22, and receiver/recorder 6 may be capable of performing the process of updating and/or replacing operational hardware configuration file 22 in a controllable manner and to minimize risk of damage to receiver/recorder 6 as a result of the process.

Sensing device 4 may include at least an imaging system 24, a control block 26, a transmitter 28, an optional receiver 30, and an antenna 32. In addition, sensing device 4 may include a power source 34 to provide power to at least imaging system 24, control block 26, transmitter 28, and optional receiver 30.

A non-exhaustive list of examples of power source 34 includes batteries, such as, for example, silver oxide batteries, Lithium batteries, capacitors, or any other suitable power source. In another embodiment of the present invention, power source 34 may not be present and the device may be powered by an external power source, for example, by a magnetic field or electric field that transmits to the device.

Imaging system 24 may include an optical window 36, at least one illumination source 38, such as, for example, a light emitting diode (LED), an imaging sensor 40, and an optical system 42.

A non-exhaustive list of examples of imaging sensor 40 includes a solid state imaging sensor, a complementary metal oxide semiconductor (CMOS) imaging sensor, a charge coupled device (CCD) imaging sensor, a linear imaging sensor, a line imaging sensor, a full frame imaging sensor, a “camera on chip” imaging sensor, or any other suitable imaging sensor.

Control block 26 may control, at least in part, the operation of sensing device 4. For example, control block 26 may synchronize time periods, in which illumination source 38 produce light rays, time periods, in which imaging sensor 40 captures images, and time periods, in which transmitter 28 transmits the images. In addition, control block 26 may produce timing signals and other signals necessary for the operation of transmitter 28, optional receiver 30 and imaging sensor 40. Moreover, control block 26 may perform operations that are complimentary to the operations performed by other components of sensing device 4, such as, for example, image data buffering.

Control block 26 may include any combination of logic components, such as, for example, combinatorial logic, state machines, controllers, processors, memory elements, and the like.

Control block 26, transmitter 28, optional receiver 30 and imaging sensor 40 may be implemented on any suitable combination of semiconductor dies. For example, and although the invention is not limited in this respect, control block 26, transmitter 28 and optional receiver 30 may be parts of a first semiconductor die, and imaging sensor 40 may be a part of a second semiconductor die. More over, such a semiconductor die may be an application-specific integrated circuit (ASIC) or may be part of an application-specific standard product (ASSP). According to some embodiments semiconductor dies may be stacked. According to some embodiments some or all of the components may be on the same semiconductor die.

Illumination source 38 may produce light rays 44 that may penetrate through optical window 36 and may illuminate an inner portion 46 of a body lumen. A non-exhaustive list of examples of body lumens includes the gastrointestinal (GI) tract, a blood vessel, a reproductive tract, or any other suitable body lumen.

Reflections 50 of light rays 44 from inner portion 46 of a body lumen may penetrate optical window 36 back into sensing device 4 and may be focused by optical system 42 onto imaging sensor 40. Imaging sensor 40 may receive the focused reflections 50, and in response to an image capturing command 52 from control block 26, imaging sensor 40 may capture an image of inner portion 46 of a body lumen. Control block 26 may receive the image of inner portion 46 from imaging sensor 40 over wires 54, and may control transmitter 28 to transmit the image of inner portion 46 through antenna 32 into wireless medium 10.

Sensing device 4 may passively or actively progress along an axis of a body lumen. In time intervals that may or may not be substantially equal and may or may not be related to that progress, control block 26 may initiate capturing of an image by imaging sensor 40, and may control transmitter 28 to transmit the captured image. Consequently, a stream of images of inner portions of a body lumen may be transmitted from sensing device 4 into wireless medium 10.

Sensing device 4 may transmit captured images embedded in “wireless communication frames”. A payload portion of a wireless communication frame may include a captured image and may include additional data, such as, for example, telemetry information and/or cyclic redundancy code (CRC) and/or error correction code (ECC). In addition, a wireless communication frame may include an overhead portion that may contain, for example, framing bits, synchronization bits, preamble bits, and the like.

Optional receiver 30 may be able to receive wireless messages via wireless medium 10 through antenna 32, and control block 26 may be able to capture these messages. A non-exhaustive list of examples of such messages includes activating or de-activating image capturing by sensing device 4, controlling the time intervals for capturing images, activating or de-activating transmissions from sensing device 4, or any other suitable messages.

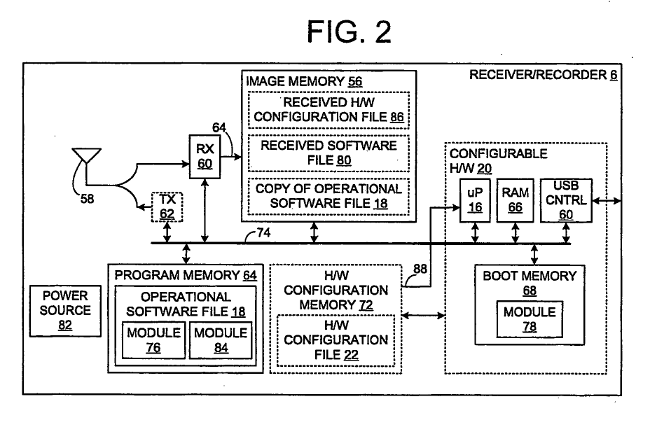

FIG. 2 is an exemplary simplified block-diagram illustration of receiver/recorder 6, in accordance with some embodiments of the invention.

Receiver/recorder 6 may include an image memory 56, an antenna 58, a receiver (Rx) 60, an optional transmitter (TX) 62, a program memory 64, a random access memory (RAM) 66, boot memory 68, and a communication controller, such as, for example, a universal serial bus (USB) controller 70. According to other embodiments of the invention, transmitter 62 may be a unit separate from receiver/recorder 6. Program memory 64 may store operational software file 18.

In addition, receiver/recorder 6 may optionally contain configurable hardware 20 and a configuration memory 72 that may store operational hardware configuration file 22, to configure configurable hardware 20.

RAM 66, boot memory 68, uP 16 and USB controller 70 may be configurable parts of configurable hardware 20. It may be appreciated that this is merely an example, and configurable hardware 20, if exists in receiver/recorder 6, may include any other combination of hardware parts of receiver/recorder 6.

Processor 16 may be able to control the operation of receiver 60, optional transmitter 62, and USB controller 70 through, for example, a bus 74. In addition, receiver 60, optional transmitter 62, processor 16 and USB controller 70 may be able to exchange data, such as, for example, images received from sensing device 4, or portions thereof, over bus 74. It may be appreciated, that other methods for control and data exchange are possible, and are under the scope of the invention.

Antenna 58 may be mounted inside or outside receiver/recorder 6, and both receiver 60 and optional transmitter 62 may be coupled to antenna 58. Optional transmitter 62 may be able to transmit wireless messages to sensing device 4 through antenna 58. Receiver 60 may be able to receive transmissions, such as, for example, a stream of wireless communication frames, from sensing device 4 through antenna 58.

Selected bits of wireless communication frames received by receiver 60 may be stored in image memory 56.

Receiver/recorder 6 may communicate with work station 8 via medium 12. For example, receiver/recorder 6 may be able to transfer bits of wireless communication frames that are stored in image memory 56 to work station 8, and may be able to receive controls, and other digital content, from work station 8. Although the invention is not limited in this respect, medium 12 may be, for example, a USB cable and may be coupled to USB controller 60 of receiver/recorder 6. Alternatively, medium 12 may be wireless, and receiver/recorder 6 and workstation 8 may communicate wirelessly.

Software file 18 may include a module 76, implementing a part of a method to replace software file 18 in program memory 64 with another file. In addition, boot memory 68 may include a module 78, implementing another part of a method to replace software file 18 in program memory 64 with another file.

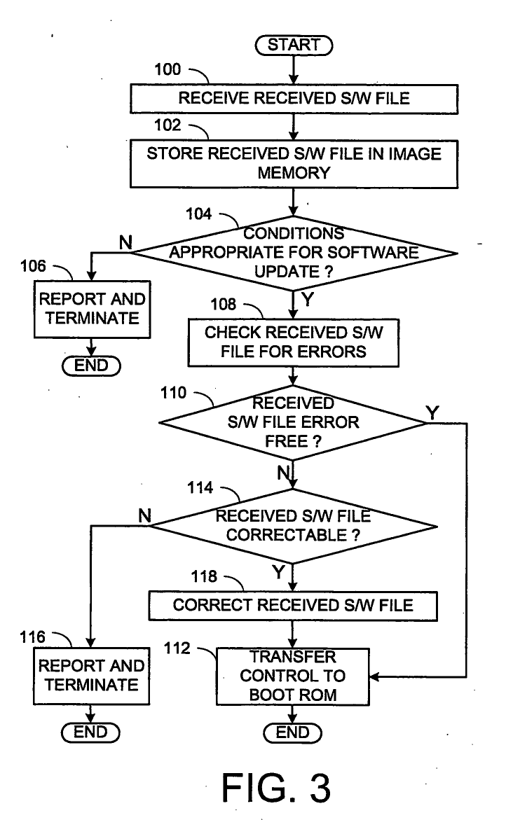

Reference is now made to FIG. 3, which is a simplified flow chart illustration of a method to replace operational software file 18 in program memory 64, in accordance with some embodiments of the invention. The method described in FIG. 3 may be implemented, for example, in module 76.

Receiver/recorder 6 may receive from work station 8 via USB controller 60 a received software file 80 (100), for example, to replace operational software file 18 in program memory 64, and uP 16 may store received software file 80 in image memory 56 (102). uP 16 may check whether conditions to replace operational software file 18 in program memory 64 with another file are fulfilled (104). For example, power for the operation of receiver/decoder 6 may be supplied, at least in part, by a power source 82 that may be, for example, a battery. uP 16 may check, for example, whether energy stored in power source 82 is sufficient to support the execution of replacing operational software file 18 in program memory 64.

If the conditions checked by uP 16 are not filled, uP 16 may report so (106), for example, to work station 8, and the method may terminate. Otherwise, uP 16 may check received software file 80 for errors (108), using for example, cyclic redundancy code (CRC) or error correction code (ECC) that may be embedded in received software file 80.

If received software file 80 is free of errors (110), or if the number of errors is correctable uP 16 may execute an instruction of module 76 that may cause uP 16 to start executing instructions of module 78 (112). The part of the method described in FIG. 3 method may then terminate.

If received software file 80 is not free of errors, uP 16 may check whether received software file 80 is correctable (114). For example, received software file 80 may be correctable if the number of errors in received software file 80 is low enough for the ECC to correct. If received software file 80 is not correctable, uP 16 may report so (116), for example, to work station 8, and the method may terminate. However, if received software file 80 is correctable, uP 16 may correct received software file 80 (118) and the method may continue to transfer control to boot ROM (112).

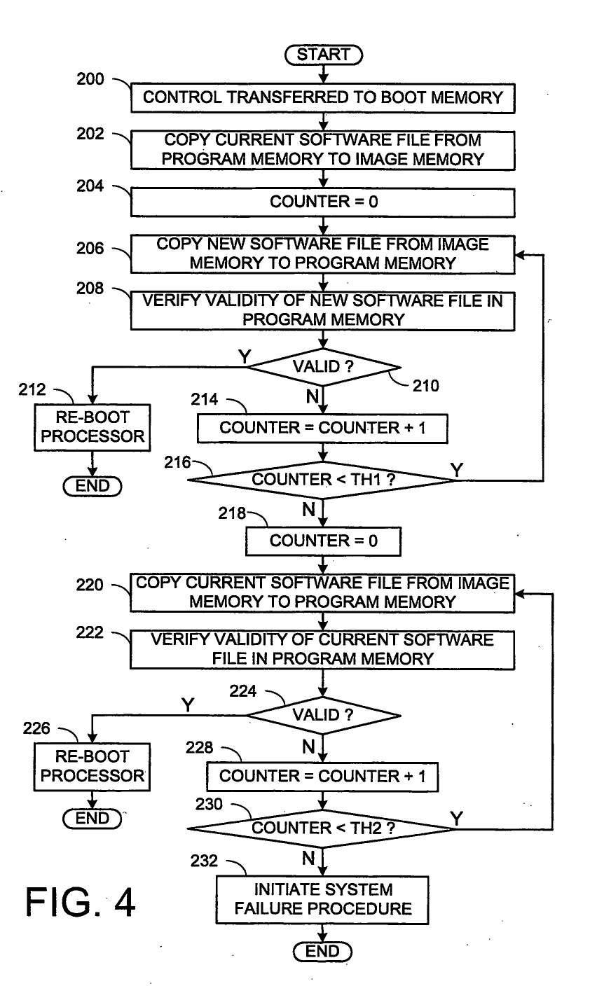

Reference is now made to FIG. 4, which is a simplified flow chart illustration of another method to replace operational software file 18 in program memory 64, in accordance with some embodiments of the invention. The method described in FIG. 4 may be implemented, for example, in module 76.

Execution of module 78 by uP 16 may start after execution of box (112) of FIG. 3 (200). uP 16 may copy operational software file 18 from program memory 64 to image memory 56 (202), and may set a counter to, for example, zero (204). uP 16 may copy received software file 80 from image memory 56 to program memory 64 (206), and as a result, parts of operational software file 18 may be erased.

uP 16 may verify whether the copy of received software file 80 in program memory 64 is identical to the copy of received software file 80 in image memory 56 (208). If the copy of received software file 80 in program memory 64 is identical to the copy of received software file 80 in image memory 56 (210), uP 16 may initiate a re-booting procedure (212).

However, if the copy of received software file 80 in program memory 64 is not identical to the copy of received software file 80 in image memory 56, uP 16 may try several more times to copy received software file 80 from image memory 56 to program memory 64. uP 16 may advance the counter by 1 (214), and if the counter's value is smaller than a predefined threshold TH1 (216), it may repeat boxes (206), (208), (210), (214) and (216), until received software file 80 is copied successfully (210), or until the number of trials defined by TH1 is exceeded.

If uP 16 unsuccessfully tried to copy received software file 80 from image memory 56 to program memory 64, uP 16 may try to restore operational software file 18 from image memory 56 to program memory 64. uP 16 may set the counter to, for example, zero (218), and may copy operational software file 18 from image memory 56 to program memory 64 (220).

uP 16 may verify whether the copy of operational software file 18 in program memory 64 is identical to the copy of operational software file 18 in image memory 56 (222). If the copy of operational software file 18 in program memory 64 is identical to the copy of operational software file 18 in image memory 56 (224), uP 16 may initiate a re-booting procedure (226).

However, if the copy of operational software file 18 in program memory 64 is not identical to the copy of operational software file 18 in image memory 56, uP 16 may try several more times to copy operational software file 18 from image memory 56 to program memory 64. uP 16 may advance the counter by 1 (228), and if the counter's value is smaller than a predefined threshold TH2 (230), it may repeat boxes (220), (222), (224), (228) and (230), until operational software file 18 is copied successfully (224), or until the number of trials defined by TH1 is exceeded.

If uP 16 unsuccessfully tried to copy operational software file 18 from image memory 56 to program memory 64, it may execute a system-failure procedure to, for example, report to a user that receiver/recoder 6 is non operational (232).

Operational software file 18 may include a module 84, implementing a method to replace hardware configuration file 22 in hardware configuration memory 72 with another file.

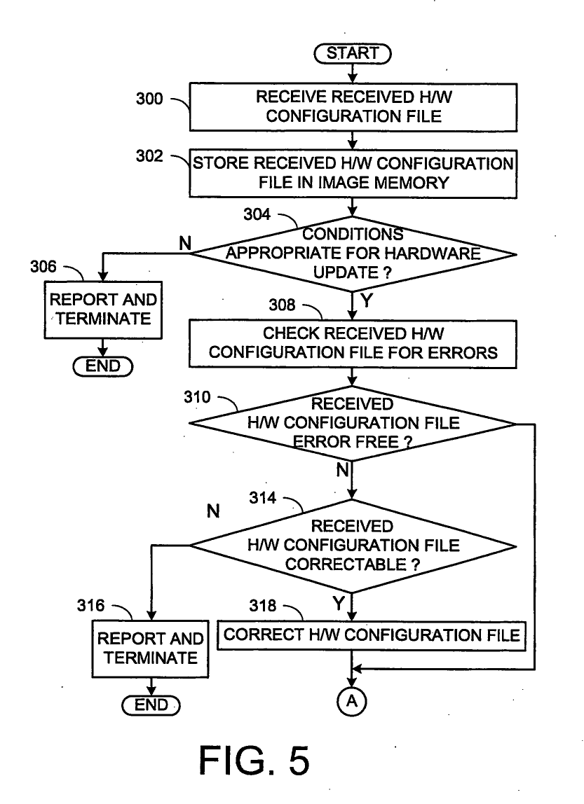

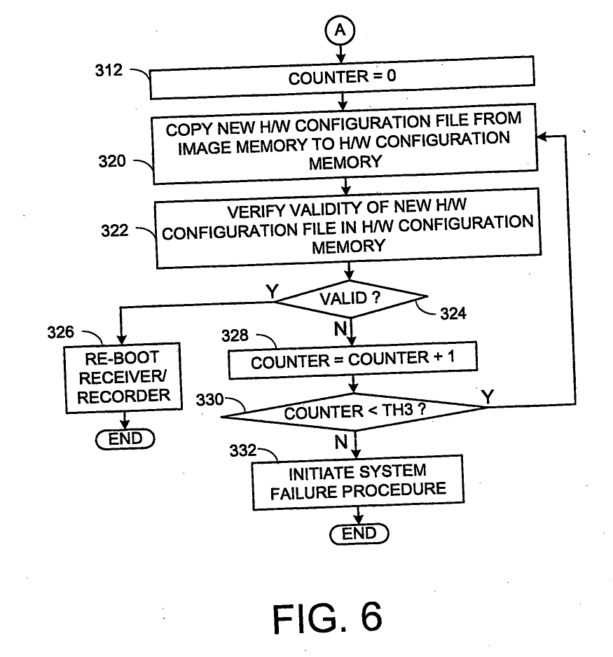

Reference is now made to FIGS. 5 and 6, which are simplified flow chart is illustrations of a method to replace hardware configuration file 22 in hardware configuration memory 72 with a received hardware configuration file 86, in accordance with some embodiments of the invention. The method described in FIGS. 5 and 6 may be implemented in module 84.

Receiver/recorder 6 may receive from work station 8 via USB controller 60 a received hardware configuration file 86 (300) to replace hardware configuration file 22 in hardware configuration memory 72, and uP 16 may store received hardware configuration file 86 in image memory 56 (302). uP 16 may check whether conditions to replace hardware configuration file 22 in hardware configuration memory 72 with another file are fulfilled (304). uP 16 may check, for example, whether energy stored in power source 82 is sufficient to support the execution of replacing hardware configuration file 22 in hardware configuration memory 72.

If the conditions checked by uP 16 are not fulfilled, uP 16 may report so (306), for example, to work station 8, and the method may terminate. Otherwise, uP 16 may check received hardware configuration file 86 for errors (308), using for example, cyclic redundancy code (CRC) or error correction code (ECC) that may be embedded in received hardware configuration file 86.

If received hardware configuration file 86 is free of errors (310) or if the number of errors is correctable, the method may continue to box (312) of FIG. 6. If received hardware configuration file 86 is not free of errors, uP 16 may check whether received hardware configuration file 86 is correctable (314). For example, received hardware configuration file 86 may be correctable if the number of errors in received hardware configuration file 86 is low enough for the ECC to correct.

If received hardware configuration file 86 is not correctable, uP 16 may report so (316), for example, to work station 8, and the method may terminate. However, if received hardware configuration file 86 is correctable, uP 16 may correct received hardware configuration file 86 (318) and the method may continue to box (312).

Reference is now made in addition to FIG. 6. uP 16 may set a counter to, for example, zero (312), and may copy received hardware configuration file 86 from image memory 56 to hardware configuration memory 72 (320). As a result, hardware configuration file 22 in hardware configuration memory 72 may be erased.

uP 16 may verify whether the copy of received hardware configuration file 86 in hardware configuration memory 72 is valid, for example, by checking an indication 88 generated by hardware configuration memory 72 (322). If the copy of received hardware configuration file 86 in hardware configuration memory 72 is valid (324), uP 16 may initiate a re-booting procedure (326).

However, If the copy of received hardware configuration file 86 in hardware configuration memory 72 is not valid, uP 16 may try several more times to copy received hardware configuration file 86 from image memory 56 to hardware configuration memory 72. uP 16 may advance the counter by 1 (328), and if the counter's value is smaller than a predefined threshold TH3 (330), it may repeat boxes (320), (322), (324), (328) and (330), until hardware configuration memory 72 is copied successfully (324), or until the number of trials defined by TH3 is exceeded.

If uP 16 failed to copy received hardware configuration file 86 from image memory 56 to hardware configuration memory 72, uP 16 may execute a system-failure procedure to, for example, report to a user that receiver/decoder 6 is non operational (332).

Image memory 56 and program memory 64 may be fixed in or removable from receiver/recorder 6. Image memory 56 may be part of program memory 64 by using simultaneous operation. A non-exhaustive list of examples of image memory 56 and program memory 64 includes any combination of the following: semiconductor devices such as registers, latches, electrically erasable programmable read only memory devices (EEPROM), not AND (NAND) flash memory devices, not OR NOR) flash memory devices, non-volatile random access memory devices (NVRAM), synchronous dynamic random access memory (SDRAM) devices, RAMBUS dynamic random access memory (RDRAM) devices, double data rate (DDR) memory devices, static random access memory (SRAM), universal serial bus (USB) removable memory, PCMCIA memory CANS, and the like; optical devices, such as compact disk read-write memory (CD ROM), and the like; and magnetic devices, such as a hard disk, a floppy disk, a magnetic tape, and the like.

A non-exhaustive list of examples of processor 16 includes a micro-controller, a micro, processor, a central processing unit (CPU), a digital signal processor (DSP), a reduced instruction set computer (RISC), a complex instruction set computer (CISC), and the like. Moreover, processor 16 may be part of an application specific integrated circuit (ASIC), may each be a part of an application specific standard product (ASSP), may be part of a field programmable gate array (FPGA), or may be a part of a complex programmable logic devices (CPLD).

A non-exhaustive list of examples of configurable hardware 20 includes any combination of FPGA devices, CPLD devices, and the like.

A non-exhaustive list of examples of hardware configuration memory 72 includes EEPROM, NVRAM, flash memory, and the like.

A non-exhaustive list of examples of antennae 26 and 58 includes dipole antennae, monopole antennae, multilayer ceramic antennae, planar inverted-F antennae, loop antennae, shot antennae, dual antennae, omni-directional antennae, coil antennae or any other suitable antennas. Moreover, antenna 32 and antenna 58 may be of different types.

A non-exhaustive list of examples of work station 8 includes a original equipment manufacturer (OEM) dedicated work station, a desktop personal computer, a server computer, a laptop computer, a notebook computer, a hand-held computer, and the like.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the spirit of the invention.

What is claimed is:

1. A method for updating an in vivo sensing system recorder, the method comprising:
replacing an operational file of a receiver of an in-vivo sensing system with a received file, said received file being received from an apparatus that is external to said receiver.

2. The method of claim 1, wherein said operational file is an operational hardware configuration file and said received file is a received hardware configuration file, and replacing said operational file with said received file further comprising:
storing said received file in a hardware configuration memory.

3. The method of claim 1, wherein said operational file is an operational software file and said received file is a received software file, and replacing said operational file with said received file further comprising:
storing said received file in a program memory.

4. The method of claim 1, further comprising:
storing said received file in a temporary storage prior to replacing said operational file with said received file.

5. The method of claim 1, further comprising:
correcting errors in said received file prior to replacing said operational file with said received file.

6. The method of claim 1, further comprising:
deciding to replace said operational file with said received file, prior to replacing said operational file with said received file.

7. The method of claim 6, comprising:
confirming that a power source of said receiver stores energy that is sufficient for replacing said operational file with said received file.

8. The method of claim 6, comprising:
confining that said received file is free of errors.

9. A method comprising:
deciding whether to replace an operational file of a receiver of an in-vivo sensing system with a received hardware configuration file, said received file being received from an apparatus that is external to said receiver.

10. The method of claim 9, further comprising:
checking whether a power source to said receiver/recorder stores energy that is sufficient for replacing said operational file with said received file prior to deciding whether to replace said operational file with said received file.

11. The method of claim 9, further comprising:
checking whether said received file is free of errors prior to deciding whether to replace said operational file with said received file.

12. The method of claim 9 comprising:
storing said received file in a hardware configuration memory.

13. An article comprising a computer-readable storage medium having stored thereon instructions that, when executed by a processor, result in:
deciding whether to replace an operational file of a receiver of an in-vivo sensing system with a received file, said received file being received from an apparatus that is external to said receiver.

14. The article of claim 13, wherein the instructions further result in:
checking whether a power source of said receiver stores energy that is sufficient for replacing said operational file with said received file prior to deciding whether to replace said operational file with said received file.

15. The article of claim 13, wherein the instructions further result in:
checking whether said received file is free of errors prior to deciding whether to replace said operational file with said received file.

16. The article of claim 13, wherein said operational file is an operational hardware configuration file and said received file is a received hardware configuration file, and the instructions further result in:
storing said received file in a hardware configuration memory.

17. The article of claim 13, wherein said operational file is an operational software file and said received file is a received software file, and the instructions further result in:
storing said received file in a program memory.

18. An in vivo sensing system comprising:
a sensing device configured for being inserted in a human body;
a receiver configured to receive sensed data from the sensing device and to receive a received file;
a workstation configured for sending the received file to the receiver; and
a processor configured to check whether a condition for replacing an operational file with the received file is fulfilled.

19. The system of claim 18 wherein the receiver comprises an operational file memory and a received file memory.

20. The system of claim 18 comprising a power source connected to the receiver and to the processor.

21. The system of claim 18 wherein the sensing device comprises an imager.

22. The system of claim 21 wherein the receiver receives images data from the sensing device.

23. The system of claim 18 wherein the sensing device comprises a wireless data transmitter.
